(19) United States
(12) Patent Application Publication
(10) Pub. No.: US 2017/0231821 A1
(43) Pub. Date: Aug. 17, 2017

(54) SILICONE WOUND CONTACT LAYER WITH SILVER

(71) Applicant: KCI USA, INC., San Antonio, TX (US)

(72) Inventors: DEBORAH ADDISON, Lancaster (GB); Rachel Hadley, Earby (GB); Paul Garvey, Beeston (GB); Sally Stephens, Skipton (GB)

(21) Appl. No.: 15/408,169

(22) Filed: Jan. 17, 2017

Related U.S. Application Data
(60) Provisional application No. 62/280,494, filed on Jan. 19, 2016.

(51) Int. Cl. A61F 13/00; A61F 13/02; A61F 15/00; B05D 3/06

(52) U.S. Cl. CPC A61F 13/00063; B05D 3/068; A61F 13/024; A61F 13/0259; A61F 15/001; A61F 13/0216; A61F 13/00072

(57) ABSTRACT

A silver-containing tissue dressing for providing antimicrobial properties to a wound site is disclosed. In some embodiments, the tissue dressing may include a wound contact layer having a substrate material coated with a tacky hydrophobic silicone material, and silver-containing particles dispersed in the silicone material. The different surfaces of the substrate material may be coated with the silicone material having various amounts of tackiness. The silver material may provide antimicrobial properties when the tissue dressing is placed in contact with the wound site.

### RELATED APPLICATION

The present invention claims the benefit, under 35 USC §119(e), of the filing of U.S. Provisional Patent Application Ser. No. 62/280,494, entitled “A Silicone Wound Contact Layer With Silver,” filed Jan. 19, 2016. This provisional application is incorporated herein by reference for all purposes.

### TECHNICAL FIELD

The invention set forth in the appended claims relates generally to tissue treatment systems and more particularly, but without limitation, to wound dressings comprising a silicone material and silver antimicrobial agents.

### BACKGROUND

Dressing materials for application to the surface of wounds should desirably be non-adherent to the moist wound surface, but sufficiently tacky to allow attachment of the dressing to intact skin around the wound and attachment to further dressing layers such as absorbent layers.

Traditional “tulle gras” dressings generally consist of a layer of gauze coated with paraffin wax. Such dressings have a number of desirable properties, and for this reason have been used extensively for many years. Among these advantages are their high degree of conformability and deformability, and the fact that their tackiness makes them very easy to apply. That is to say, a tulle gras dressing applied to a wound will usually remain in place simply by adhesion of the paraffin wax to the patient's skin (or to itself in the case of a dressing wrapped around a finger, for example) while a securing bandage is applied. Tulle gras dressings are also quite inexpensive. However, tulle gras dressings do have a number of disadvantages. Principal amongst these is that, although initially non-adherent, they often become “dry” (in the sense of losing their paraffin coating) and consequently adhere to the wound to which they are applied. This effect is due to the paraffin coating becoming mobile at body temperatures and migrating into the wound or being absorbed into the backing of the dressing or bandage. In some cases, removal of a tulle gras dressing which has become dry in this way can cause considerable trauma. Indeed, it is quite common to have to soak tulle gras dressings in order to remove them. If tulle gras dressings are changed more frequently, in an attempt to avoid them becoming attached to the wound, this may delay wound healing and adds to nursing costs.

A further disadvantage of traditional tulle gras dressings is that fibers from the gauze may become incorporated in the wound, as may the paraffin coating of the dressing. Some authorities see the migration of paraffin into a wound as an undesirable effect and any paraffin found in a wound can be difficult to remove with normal aqueous wound-cleansing agents. Moreover, the pores of the gauze may become occluded if the paraffin coating is too heavy or as a result of the mobility of the paraffin during use of the dressing. While occlusive dressings are appropriate in some circumstances, it is undesirable that the nursing staff should have no control over whether the dressing used is in fact occlusive.

Still further disadvantages of conventional tulle gras dressings are that they are effectively opaque and of somewhat unsightly appearance, and the paraffin can run during storage, making them particularly messy to apply.

EP-A-0251810 describes wound dressing materials that overcome the above disadvantages by replacing the paraffin wax coating of conventional tulle gras by a tacky or non-tacky, hydrophobic silicone coating on a gauze or mesh substrate. In certain embodiments, the gauze may be provided with a tacky silicone coating on one side and a non-tacky silicone coating having a different composition on the other side. Similar materials are described in WO-A-8705206.

EP-A-0342950 describes similar wound dressings having a non-adherent silicone coating. The adherence of the silicone is reduced by addition of an amine-extended polyurethane.

U.S. Pat. No. 6,846,508 describes medical adhesive devices having tacky silicone layers on both surfaces of a substrate. The material of the tacky layers may be the same or different, and the tackiness of the layers may be the same or different.

U.S. Pat. No. 5,891,076 describes scar dressings comprising a carrier material embedded in a layer of silicone gel which is tacky, and cover sheets over the silicone gel layers.

JP-A-10095072 describes a double-sided silicone adhesive tape for non-medical adhesive applications and having release sheets over the silicone adhesive.

While the use of silicone in wound dressings is known, the cost and complexity of wound treatment can be a limiting factor in the use of these dressings, and the development and application of new silicone wound dressings continues to present significant benefits to healthcare providers and patients.

### BRIEF SUMMARY

New and useful systems, apparatuses, and methods for providing a silver-containing tissue dressing are set forth in the appended claims. Illustrative embodiments are also provided to enable a person skilled in the art to make and use the claimed subject matter.

For example, in some embodiments, wound dressings comprising a substrate coated on upper and lower surfaces with a tacky, silicone gel including a silver-containing compound, are disclosed. In some embodiments, the upper and lower surfaces of the substrate may have different amounts of tackiness.

In some embodiments, a wound dressing product may include a substrate layer having an upper surface and a lower surface, a tacky silicone coating composition applied to the upper surface and lower surface, a silver material disposed in the tacky silicone coating composition, and upper and lower release sheets. The upper release sheet may be adapted to cover the tacky silicone coating composition on the upper surface of the substrate layer, and the lower release sheet may be adapted to cover the tacky silicone coating composition on the lower surface of the substrate layer. In some embodiments, the upper surface may be more tacky than the lower surface.

Additionally, other example embodiments may include a method of making a wound dressing material. The method of making a wound dressing material may include providing a substrate layer having an upper surface and a lower surface, coating the upper and lower surfaces of the substrate layer with a fluid silicone prepolymer composition comprising a silver material, thermally partially curing the silicone prepolymer composition to produce an intermediate material having a partially cured silicone composition on the upper and lower surfaces, and further curing the partially-cured silicone composition by exposing the intermediate material to ionizing radiation to produce a final material having tacky silicone coatings on the upper surface and the lower surface. The step of coating the upper and lower surfaces of the substrate layer may comprise applying unequal weights of the silicone coating composition to the upper surface and the lower surface. The method of making may further include storing the intermediate material at a temperature below 50° C. for at least two days to allow equilibration of the silicone coatings on the upper surface and the lower surface.

Another example embodiment of a method of making a wound dressing material is also described herein, wherein the example embodiment may include providing a substrate layer having an upper surface and a lower surface, coating the upper surface and the lower surface of the substrate layer with a fluid silicone prepolymer composition comprising a silver material, thermally partially curing the silicone prepolymer composition to produce an intermediate material having a partially-cured silicone composition on the upper surface and the lower surface, and further curing the partially-cured silicone composition by exposing the intermediate material to ionizing radiation to produce a final material having tacky silicone coatings on the upper surface and the lower surface. The step of thermally partially curing the silicone prepolymer composition may apply different amounts of heat to the upper surface and the lower surface, whereby the silicone coatings on the upper surface and the lower surface may have different levels of tackiness following the further curing step.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a schematic diagram showing a perspective exploded view of an embodiment of a dressing;

FIG. 2 is a schematic diagram showing an enlarged partial cross-section view of the dressing of FIG. 1;

FIG. 3 is a schematic view of an apparatus for making a dressing;

FIG. 4 shows a schematic diagram of the apparatus used for the loop tack measurement test;

FIG. 5 is a schematic diagram showing a perspective view of a dressing packaged in a microorganism-impermeable pouch;

FIG. 6 is a graphical view showing experimental results of an embodiment of a dressing; and

FIG. 7 is a graphical view showing experimental results of an embodiment of a dressing.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

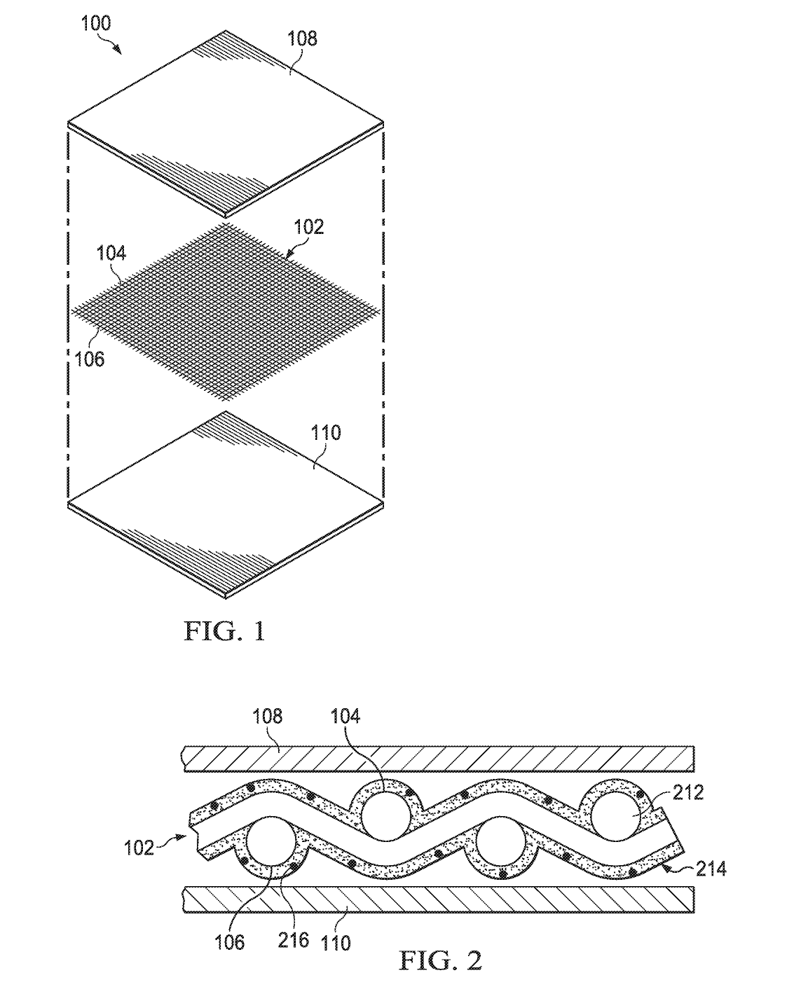

FIG. 1 is a perspective view illustrating details that may be associated with a silver-containing tissue dressing 100. The tissue dressing 100 according to the invention comprises a wound contact layer 102 having an upper surface 104 and a lower surface 106. The tissue dressing 100 may further include an upper release sheet 108 and a lower release sheet 110, which may respectively cover the upper surface 104 and the lower surface 106 of the wound contact layer 102. The upper surface 104 and the lower surface 106 of the wound contact layer 102 may include a tacky silicone coating composition, and the upper release sheet 108 and the lower release sheet 110 may be adhered to the upper surface 104 and the lower surface 106, respectively, by the tacky silicone coating composition. In some embodiments, the upper surface 104 of the wound contact layer 102 may be more tacky than the lower surface 106 of the wound contact layer 102, which may allow the lower release sheet 110 to be removed from the lower surface 106 more readily than the upper release sheet 108 may be removed from the upper surface 104.

The release sheets, such as upper release sheet 108 and lower release sheet 110, may include a film of polyethylene, polypropylene, or fluorocarbons, and papers coated with these materials. In some preferred embodiments, the release sheets may be release-coated paper sheets, such as silicone release-coated paper sheets. Examples of silicone-coated release papers are POLYSLIK (Registered Trade Mark) supplied by H.P. Smith & Co., offered in various formulations to control the degree of adhesion of the paper to the adhesive surface.

In certain embodiments, one or both release sheets may comprise two or more parts, such as a first removable part having a first edge and a second removable part that meets the first part along the first edge. Suitably, along each of said edges where the parts meet, one of the parts may be folded back to provide a folded-back margin, and the other part may overlap the said folded-back margin. This may provide an easy-to-grasp margin on each part in the region of overlap to assist with removal of the release sheet by the caregiver. In some embodiments, one or both release sheets may comprise three parts, for example as described in detail in EP-A-0117632. The tissue dressing 100 may be applied to the surface of a wound by removing the release sheets, such as upper release sheet 108 and lower release sheet 110.

Referring primarily now to FIG. 2, the wound contact layer 102 may include a substrate material 212, which in some embodiments may be a porous material that is permeable to fluid. For example, the substrate material 212 may be a web or fabric formed from a woven, nonwoven, or knitted textile, or a molded mesh. The substrate material 212 may be suitably formed from any medically-acceptable material, such as cellulose, polyolefins, polyesters, or polyamides. An especially suitable material may be cellulose acetate gauze. Substrate materials having a weight of from 15 to 200 g/m² are generally found to be suitable for use in the wound contact layer 102, and fabrics weighing from 50 to 150 g/m² may be most suitable. For example, some preferred embodiments may include a fabric of from 80 to 120 g/m². In some embodiments, the substrate material 212 may be a cellulose acetate gauze of density 107 g/m² nominal.

In this example, the upper surface 104 and the lower surface 106 of the wound contact layer 102 are coated with a tacky silicone composition 214, such as the silicone composition described in U.S. Patent Application Publication No. US 2013/0165837 A1, which is hereby incorporated by reference in its entirety. In some embodiments, the substrate material 212 may be permeable to the silicone composition.

The wound contact layer 102 may further include a silver material 216, such as silver particles, ionic silver, and silver salts, for providing antimicrobial and/or bacteriostatic properties. The silver material 216 may be distributed throughout, as well as on the surface of, the wound contact layer 102. For example, in some embodiments, the silver material 216 may include silver acetate, which may be added to the silicone composition 214. In some embodiments, the silver material 216 may also include other silver compounds, such as silver orthophosphate, silver sulfate, silver sodium hydrogen zirconium phosphate, silver lactate, silver-oxidized regenerated cellulose (ORC), and silver chloride.

In some embodiments, the tacky silicone coating composition, such as silicone composition 214, is a soft skin adhesive silicone elastomer. Such silicone coating compositions can be made by an addition reaction (hydrosilylation) between (a) a vinyl functional polydimethyl siloxane, such as bis-dimethyl vinyl PDMS, and (b) a hydrogen functional siloxane, such as dimethyl, methylhydrogen siloxane copolymers, hydrogen dimethylsiloxy terminated PDMS. The cure reaction may be catalyzed by a hydrosilylation catalyst, such as a noble metal catalyst, suitably a platinum catalyst. The silicone prepolymer composition may further comprise a polymerization inhibitor, for example 2-methyl-3-butyn-2-ol, which may be evaporated from the composition during a step of thermally partially curing. The polymerization inhibitor may be present in an amount of from about 0.001 wt. % to about 1 wt. %, for example from about 0.01 wt. % to about 0.1 wt. % before curing.

Silicone coating compositions may be supplied as two-part systems: Part A may contain at least the vinyl pre-polymer and the catalyst, while Part B may contain the vinyl pre-polymer and the SiH siloxane cross linker. The components should be mixed immediately before use, optionally with addition of the polymerization inhibitor. In some embodiments, the silver material 216, such as silver particles, may be added to either Part A or Part B prior to mixing.

In some embodiments, the silicone coating composition may comprise or consist essentially of the following components:
(A) a diorganopolysiloxane having at least 2 alkenyl groups in each molecule;
(B) an organohydrogenpolysiloxane having at least 2 silicon-bonded hydrogen atoms in each molecule, in a quantity sufficient for the ratio between the number of moles of silicon-bonded hydrogen atoms in this component and the number of moles of alkenyl groups in component (A) to have a value of from about 0.6:1 to about 20:1;
(C) optionally a platinum group metal catalyst suitably in a quantity providing 0.1 to 500 weight parts as platinum group metal per 1,000,000 weight parts component (A); and
(D) a volatile polymerization inhibitor, suitably selected from: alkyne alcohols such as 20 2-methyl-3-butyn-2-ol, 3,5-dimethyl-1-hexyn-3-ol, and phenylbutynol; ene-yne compounds such as 3-methyl-3-penten-1-yne and 3,5-dimethyl-3-hexen-1-yne; tetramethyltetrahexenyl-cyclotetrasiloxane; and benzotriazole.

The diorganopolysiloxane, component (A), may be the base component of the total composition. This diorganopolysiloxane must contain at least 2 alkenyl groups in each molecule in order for this composition to cure into a rubbery elastic silicone rubber coating composition. The diorganopolysiloxane (A) comprises essentially straight-chain organopolysiloxane with the average unit formula RₙSiO_{(4-n)/2}, wherein R is selected from substituted and unsubstituted monovalent hydrocarbon groups and n has a value of 1.9 to 2.1. R may be exemplified by alkyl groups such as methyl, ethyl, propyl, and others; alkenyl groups such as vinyl, allyl, and others; aryl groups such as phenyl, and others; and haloalkyl groups such as 3,3,3-trifluoropropyl and others. The diorganopolysiloxane (A) should have a viscosity at 25° C. of at least 100 centipoise (1 d Pa·s). When such factors as the strength of the silicone rubber coating membrane, and blendability are taken into account, the viscosity of diorganopolysiloxane (A) at 25° C. is preferably from 1,000 centipoise (1 Pa·s) to 100,000 centipoise (100 Pa·s). The diorganopolysiloxane (A) may be exemplified by dimethylvinylsiloxy-endblocked dimethylpolysiloxanes, dimethylvinylsiloxy-endblocked dimethylsiloxane-methylvinylsiloxane copolymers, and dimethylvinyl-siloxy-endblocked dimethylxilosane-methylphenylsiloxane copolymers.

Component (B), an organopolysiloxane that contains at least 2 silicon-bonded hydrogen atoms in each molecule, is a crosslinker for the composition. The organopolysiloxane (B) may be exemplified by trimethylsiloxy-endblocked methyl-hydrogenpolysiloxanes, trimethylsiloxy-endblocked dimethylsiloxanemethylhydrogen-siloxane copolymers, dimethylphenylsiloxy-endblocked methylphenylsiloxanemethyl-hydrogensiloxane copolymers, cyclic methylhydrogenpolysiloxanes, and copolymers that contain the dimethylhydrogensiloxy unit and SiO4/2 unit. The organohydrogenpolysiloxane (B) should be added in a quantity that the ratio between the number of moles of silicon-bonded hydrogen atoms in this organohydrogenpolysiloxane and the number of moles of alkenyl groups in component (A) has a value of 0.6:1 to 20:1.

The platinum group metal catalyst, component (C), used in the compositions is a curing catalyst. The platinum group metal catalyst (C) may be exemplified by platinum micropowder, platinum black, chloroplatinic acid, platinum tetrachloride, olefin complexes of chloroplatinic acid, alcohol solutions of chloroplatinic acid, complexes between chloroplatinic acid and alkenylsiloxanes, rhodium compounds, and palladium compounds. The platinum group metal catalyst (C) should be added generally at 0.1 to 500 weight parts as platinum group metal per 1,000,000 weight parts component (A), and is preferably used at 1 to 50 weight parts as platinum group metal per 1,000,000 weight parts component (A). The reaction will not develop adequately at less than 0.1 weight parts, while additions in excess of 500 weight parts are uneconomical.

The wound contact layer 102 may desirably be liquid-permeable to allow passage of wound fluid, especially when used in the context of heavily-exuding wounds such as burns. In some embodiments, the wound contact layer 102 may include an array of apertures. In some embodiments, following the coating of the substrate material 212 with a silicone coating composition, gas, such as air, may be blown through the substrate material 212 to ensure that the apertures in the material are open after the coating. The size and shape of the apertures in the substrate material 212 are not critical, but the apertures may suitably be such as to ensure that the substrate material 212 can be adequately coated with a silicone coating composition without them becoming occluded. In some embodiments, the apertures may generally have an aspect ratio of from 1:1 to 5:1, and preferably from 1:1 to 2:1. For example, the apertures may be approximately circular or approximately square in shape. In some embodiments, the apertures may have an average diameter of from 0.3 to 4 mm, and more suitably from 0.5 to 2 mm. The open area of the coated substrate layer 102 in the final product may, for example, be from about 1% to about 70%, for example from about 10% to about 50%. The array of apertures extending through the substrate layer 102 and the silicone coating may allow passage of wound fluid through the material.

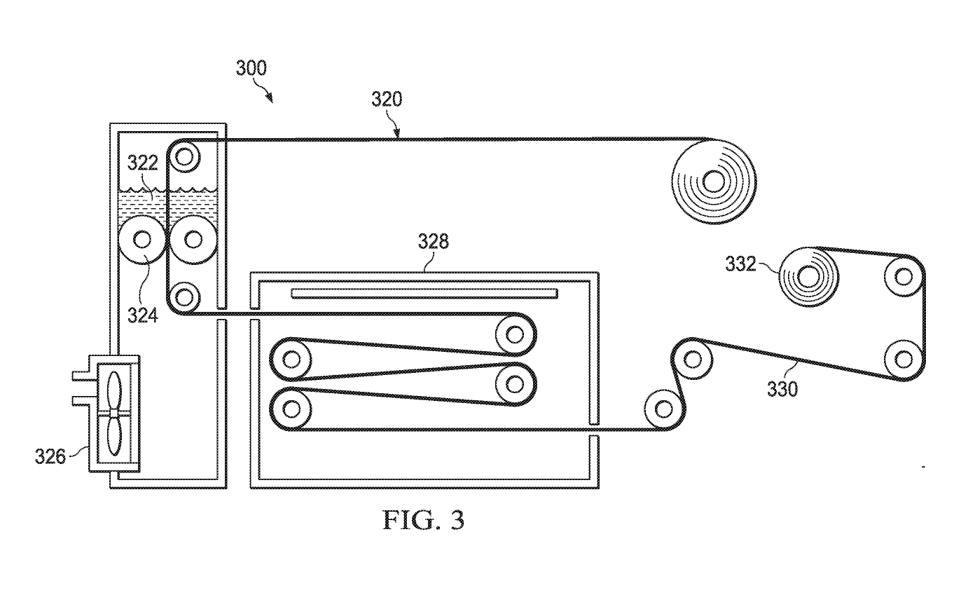

The silicone composition may penetrate the substrate material 212 to form a single, chemically homogeneous silicone phase on the upper surface 104 and the lower surface 106. The coating of the substrate material 212 with a silicone coating composition, such as silicone composition 214, may be performed in any conventional way, for example by immersion, spraying, or by using a doctor blade. For example, FIG. 3 is a schematic view of an apparatus 300 as well as process for performing the coating of the substrate material 212, according to some illustrative embodiments. In some embodiments, the process may start from a continuous web of cellulose acetate gauze 320 that may be passed through a fluid silicone coating composition 322 and nip rollers 324 to coat and impregnate the cellulose acetate gauze 320 with the fluid silicone coating composition 322. The nip rollers 324 may help ensure a smooth coating and penetration of the fluid silicone coating composition 322. In some preferred embodiments, the fluid silicone coating composition 322 may be substantially or completely solvent-free. The fluid silicone coating composition 322 may be prepared by mixing Components A and B of a soft silicone skin adhesive silicone elastomer kit supplied by Dow Corning under product reference Q7-9177. The components may be mixed in weight ratio 50:50. Component A may comprise a bis-dimethylvinyl terminated polydimethylsiloxane and a platinum catalyst. Component B may comprise a bis-hydride terminated polydimethylsiloxane. Additionally, a 2-methyl-3-butyn-2-ol inhibitor at a concentration of 0.02 wt. % may be added to the mixture.

Still referring to FIG. 3, the coated substrate, such as the cellulose acetate gauze 320 that is coated with the fluid silicone coating composition 322, may then be passed over a blower 326 to open the apertures of the coated substrate that may have been occluded by the silicone coating. The coated substrate material may then be subjected to thermal curing to partially cure the silicone. The thermal coating may be performed continuously by passing the coated substrate through an oven, such as oven 328. Suitable thermal curing conditions include exposure to a temperature of from about 80° C. to about 200° C., for example about 120° C. to about 180° C. for a time of from about 1 minute to about 10 minutes, for example about 1.5 minutes to about 5 minutes. In some preferred embodiments, the coated substrate may be passed through an oven, such as oven 328, held at 150° C., for 5 passes at 4.2 m/min, with a total residence time of 1.5 minutes. The elevated temperature results in evaporation of the polymerization inhibitor from the silicone composition and therefore in polymerization of the silicone. The resulting material is chemically polymerized, as a thermal partial cure of the silicone coating has been completed, but the material is capable of further curing by ionizing radiation as explained further below. The coated material may then be allowed to cool, and release-coated paper cover sheets (not shown) may then be continuously applied to the upper and lower surfaces at location 330 and the material may be rolled up on roll 332 for equilibration.

The rolls 332 of thermally-cured and interleaved material may be allowed to equilibrate at controlled temperature (20-25° C.) for 4-6 weeks. The material may then be cut and packaged, followed by the application of gamma irradiation with 35-50 kGy of Cobalt 60 radiation at 7-9 kGy/hr to sterilize the products and complete the cure. The irradiation curing may result in a further increase in both hardness and tackiness of the silicone coating.

As discussed, in some embodiments, the partially-cured material may be subjected to a final cure with ionizing radiation. In some preferred embodiments, the release sheets may be applied intermediate the steps of thermally partially curing and storing, whereby, for example, the partially-cured material having the release sheets may be rolled up for storage. In other preferred embodiments, the partially-cured material may be packaged in a microorganism-impermeable container prior to the final cure with the ionizing radiation so that the final cure also sterilizes the microorganism-impermeable material. The ionizing radiation may be selected from e-beam radiation and gamma radiation, each of which exists in a variety of well-known procedures. The cure may depend on the specific equipment used, and those skilled in the art may define a dose calibration model for the specific equipment, geometry, and line speed, as well as other well understood process parameters.

Commercially-available electron beam generating equipment is readily available, for example, a Model CB-300 electron beam generating apparatus (available from Energy Sciences, Inc. (Wilmington, Mass.)). Generally, a support film (e.g., polyester terephthalate support film) may run through a chamber, and the chamber may be flushed with an inert gas, e.g., nitrogen, while the samples are e-beam cured. Multiple passes through the e-beam sterilizer may be needed.

Commercially-available gamma irradiation equipment includes equipment often used for gamma irradiation sterilization of products for medical applications. Cobalt 60 sources may be appropriate. Total absorbed doses may be from 20 to 60 kGy, and more preferably from about 35 to 50 kGy, and dose rates may be from about 7 to 8 kGy/hour.

Referring again to FIGS. 1 and 2, the partially-cured material may have unequal tackiness of the silicone coating on the surfaces of the substrate material, such as the upper surface 104 and the lower surface 106 of wound contact layer 102. The unequal tackiness can be produced by (1) unequal coating weights of the silicone on the upper surface 104 and the lower surface 106, and/or (2) the step of blowing air through the coated substrate to open the apertures, which results in a higher coating weight of silicone on the surface that is downstream of the air flow, and/or (3) unequal amounts of heat supplied to the upper and lower surfaces in the oven resulting in different degrees of cure on the two surfaces.

The initial difference in tackiness of the two surfaces, such as the upper surface 104 and the lower surface 106 of the substrate layer 102, may typically be rather greater than desired for the final product. However, the tackiness of the two surfaces gradually equilibrates if the partially-cured material is stored at temperatures below about 50° C., for example ambient or near-ambient temperatures such as 10-50° C., for a period of from about 2 days to about 10 weeks. Therefore, suitably the partially-cured material is stored at such temperatures for a period of at least about 2 days, suitably about 2 weeks to about 10 weeks, for example about 3 weeks to about 8 weeks, before the final cure with ionizing radiation.

As discussed above, the dressing materials of the invention are characterized by tacky upper and lower silicone coatings on the wound contact layer 102, wherein the upper and lower silicone coatings may have different levels of tackiness. This provides the advantage that both the upper surface 104 and the lower surface 106 of the wound contact layer 102 may be protected before use by release sheets, such as upper release sheet 108 and lower release sheet 110, respectively, which may be adhered to the coating by the tackiness thereof. However, one of the release sheets may be removed more easily than the other, whereby application of the dressing may be made easier. In some preferred embodiments, the lower release sheet 110 may be less strongly adhered to the wound contact layer 102 than the upper release sheet 108, but also the resulting exposed less-adherent surface of the coated wound contact layer 102 may be more suitable for application to a wound surface. The upper release sheet 108 may then be removed to expose a more adherent surface for application of secondary dressing layers, such as absorbent layers, to the upper surface 104 of the coated wound contact layer 102.

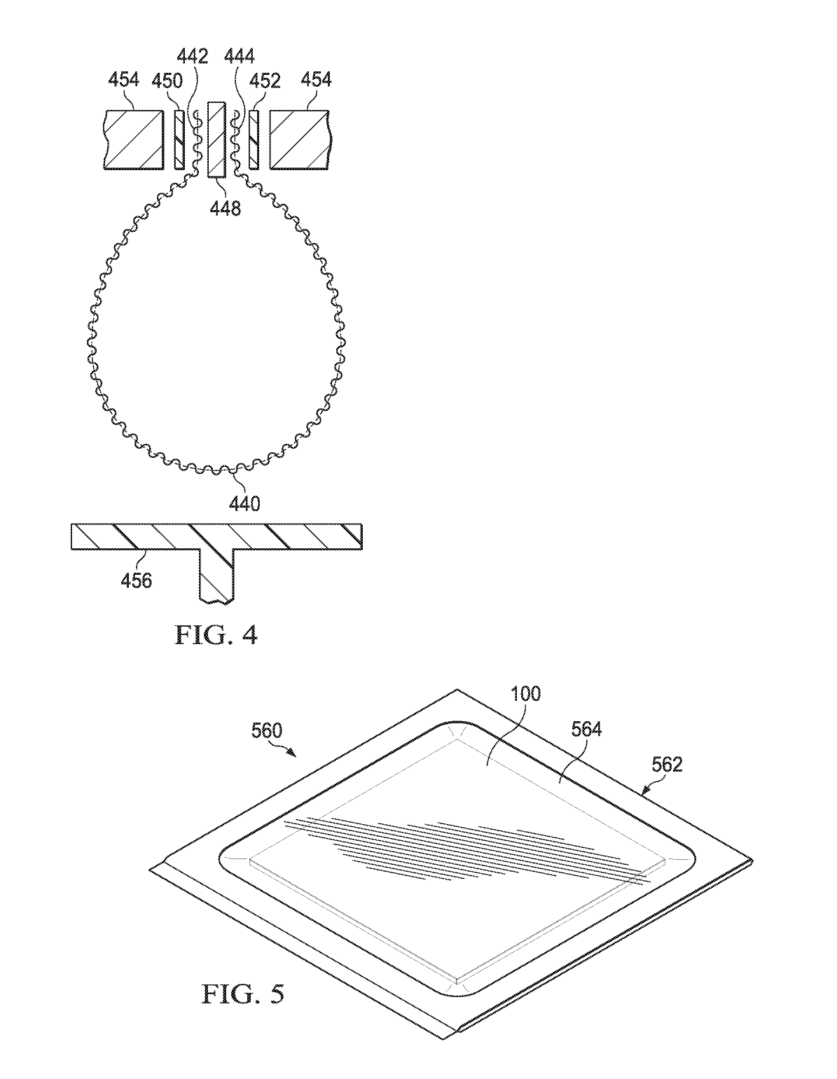

The difference in tackiness between the upper and lower silicone coatings of the wound contact layer 102 may be selected to optimize the above-described properties. The tackiness of the silicone coatings produced by the methods of the invention may be measured in a tensile tester, such as an Instron tester, using the set-up shown in FIG. 4. Samples of the coated fully cured wound contact layer 102 having the release cover sheets attached may be cut to appropriate dimensions, for example measurements of 5 cm×9.5 cm. Margins, which may be approximately 1 cm, may be marked out along the long edges by drawing straight lines 1 cm from the long edges. The cover sheets may be removed, and the wound contact layer material 440 may be looped around and the 1 cm margins 442, 444 on opposed edges of one surface (opposite the surface being measured) may be applied firmly to opposite sides of a 2 mm thick metal spacer bar 448. Strips of polypropylene film 1 cm wide 450, 452 may then be applied to the opposite surfaces of the coated gauze opposite the spacer bar 448 to prevent the coated gauze from adhering to the jaws of the measurement device.

The assembly of polypropylene strips, coated gauze, and spacer bar may then be gripped in the jaws 454 of the Instron tester. The loop of the wound contact layer material 440 having the surface under test outermost may then be lowered onto a clean polycarbonate surface 456 of dimensions 15.5 cm×3.8 cm so that the loop adheres to the surface, and raised to detach the loop from the surface. Lowering and raising may be performed at 300 mm/min, and the minimum distance between the jaws 454 and the polycarbonate surface 456 may be 15 mm. The measured tack (in Newtons) is the maximum force measured while detaching the loop from the surface. The average of three measurements may be used.

For example, the tackiness of the upper surface 104 of the wound contact layer 102, as measured by a loop tack test, may be suitably from 5% to 150% greater than the tackiness of the lower surface 106, more suitably from 20% to 100% greater, for example about 30% to 70% greater. In some preferred embodiments, the tackiness of the upper surface 104 may be approximately 50% greater than the tackiness of the lower surface 106. Suitably, the tackiness of the surfaces as measured by the loop tack test may be greater than about 0.3N. For example, the tackiness of the surfaces may be from about 0.4N to about 2N, more suitably from about 0.5N to about 1.5N. In some preferred embodiments, the tackiness of one surface is from about 0.4N to about 1N, and the tackiness of the other surface is from about 0.5N to about 1.5N.

Suitably, the above-described differences in tackiness can be achieved in materials wherein the silicone on said upper surface 104 and lower surface 106 is substantially chemically homogenous. The term “chemically homogenous” signifies that the chemical analysis (wt % silicon, carbon, oxygen, etc.) is the same on the upper and lower surfaces, but the tackiness may be different due to different degrees or type of cure between the surfaces and/or different coating thicknesses on the two surfaces. That is to say that the same silicone prepolymer may be coated onto the upper surface 104 and the lower surface 106 of the wound contact layer 102, but differences in coating or curing conditions may be used to achieve different amounts of tackiness in the upper surface 104 and the lower surface 106. Thus, both surfaces of the wound contact layer 102 may be formed by curing the same fluid silicone prepolymer. The total coating weight of the tacky silicone (combined upper and lower layers) is suitably from about 50 g/m2 to about 500 g/m2, for example from about 80 g/m2 to about 200 g/m2, typically from about 100 g/m2 to about 150 g/m2, and in some preferred embodiments from about 120 g/m2 to about 130 g/m2. In some preferred embodiments, the silicone may be a soft skin adhesive silicone composition and may be hydrophobic.

FIG. 5 is a perspective view of a silver-containing tissue dressing, such as tissue dressing 100, shown sterile and packaged into a tissue dressing product 540. The tissue dressing product 540 is shown sterile and packaged in a microorganism-impermeable envelope 542 having a transparent window 544.

In use, the tissue dressing 100 may be applied to a tissue site, which may include a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example.

When applying the tissue dressing 100 to the tissue site, the lower release sheet 110 of the tissue dressing 100 may be removed first to expose the less tacky lower surface 106 of the wound contact layer 102. It may be relatively easy to selectively remove the lower release sheet 110 because of the lower adherency of this sheet to the wound contact layer 102 compared to the upper release sheet 112. In some embodiments, the release sheets may include indicia for providing instructions to a user, such as indicia to identify the order of which the release sheets should be removed. For example, the lower release sheet 110 may comprise indicia for indicating that it should be removed first. The lower surface 106 of the wound contact layer 102 may then be applied to a tissue site, such as a wound surface, followed by removal of the upper release sheet 112 and application of secondary dressing elements, such as an absorbent layer.

Upon application of the lower surface 106 of the wound contact layer 102 to the tissue site, such as a wound, the hydrophobic soft silicone material of the wound contact layer 102 may be in intimate contact with the wound surface. As fluid from the wound, such as exudates, migrates from the wound surface on or into the silicone material of the wound contact layer 102, the fluid may come into contact and/or mobilize the silver material 216 to be released from the wound contact layer 102. Antimicrobial silver ions may dissociate from a silver compound, such as silver acetate, and be released from the wound contact layer 102 into the wound site, with some silver ions also remaining throughout the wound contact layer 102. The wound contact layer 102 may therefore provide a sustained release of low levels of ionic silver. This sustained release may reduce the risk of silver toxicity and may reduce the need for frequent dressing changes.

In some embodiments, the wound contact layer 102 may be printed or dyed with decorative or informative matter. For example, the decorative or informative matter may be printed or dyed on the substrate material 212. Due to the ability of the silicone composition to encapsulate the wound contact layer 102, there may be little danger of the ink or dye being released into the wound to which the dressing is applied. Visible indicia, such as color or writing, may be provided on one or both of the surfaces of the wound contact layer 102 and/or on one or both of the upper release sheet 108 and the lower release sheet 110 to indicate which sides of the product 100 have the more/less tacky silicone coating, and thus to indicate which silicone surface is less tacky for application to the wound surface.

The systems, apparatuses, and methods described herein may provide significant advantages. For example, the silver-containing tissue dressing described above may provide a silicone dressing cured and sterilized with gamma irradiation that contains antimicrobial silver particles. Despite previous expectations in the field that the silicone may be dry following gamma irradiation, the silver-containing silicone dressing described above may include antimicrobial properties in a tacky dressing that has been sterilized with gamma irradiation.

Furthermore, contrary to expectations in the field, the silver antimicrobial agent may be added to a hydrophobic silicon base and allow for the release of the silver antimicrobial agent from the silicone, without the addition of a hydrophilic component to the hydrophobic silicone base. The inclusion of the silver antimicrobial agent may enable silicone-containing wound dressings, such as the ADAPTIC TOUCH dressing, commercially available from Systagenix Wound Management, Inc. of San Antonio, Tex., USA, to be suitable for use on infected and colonized wounds, as silver ions have been shown to have potent antimicrobial properties against a wide range of microorganisms, including antibiotic-resistant species, even when present in low amounts. Furthermore, the physical and chemical properties of the silicone-containing wound dressing may allow for more intimate contact with a wound surface than forms of antimicrobial foam currently available, better facilitating the release of silver ions from the wound dressing into the wound.

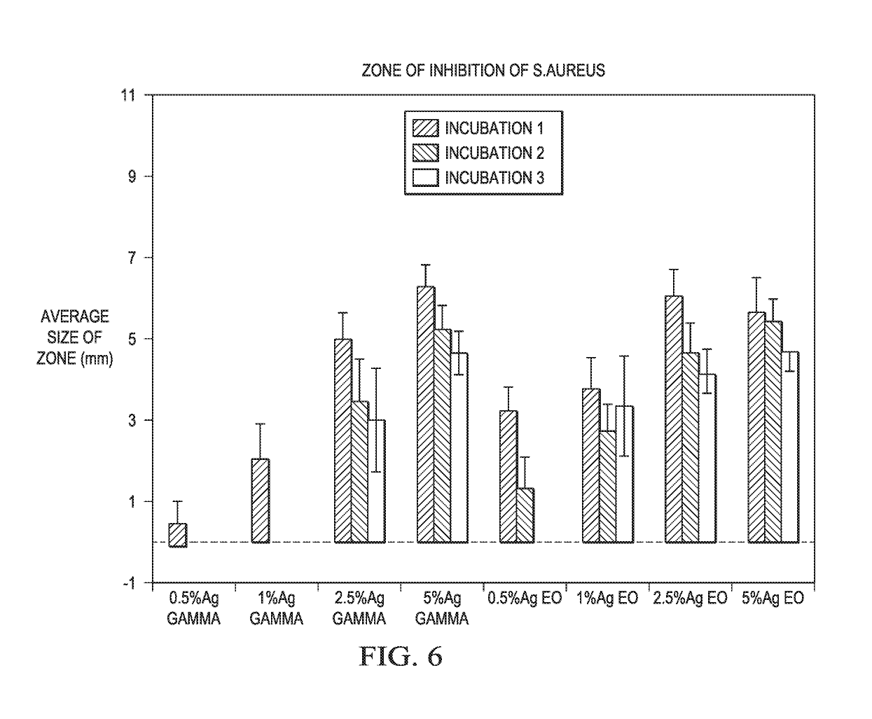

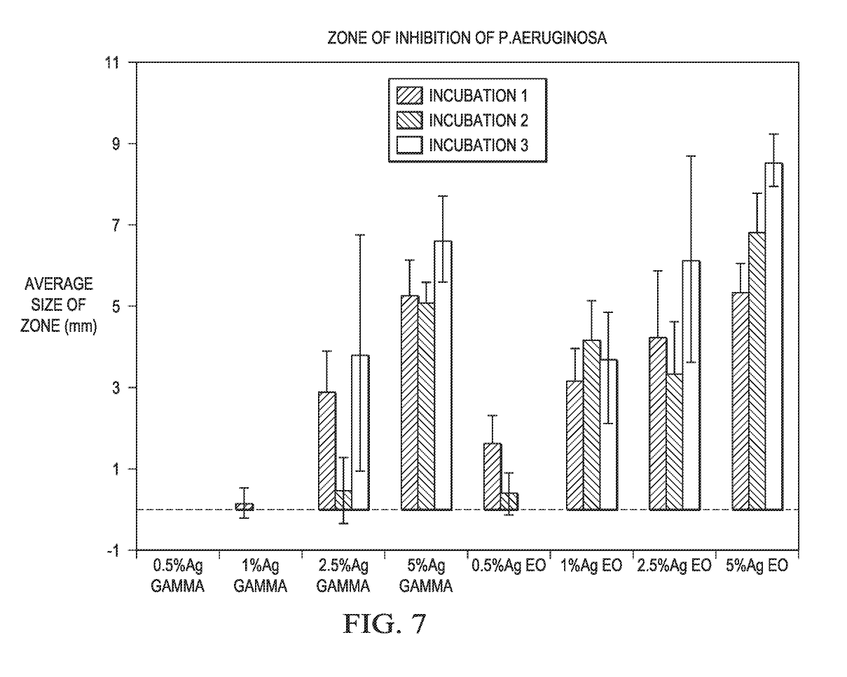

FIGS. 6 and 7 illustrate experimental results of samples of silver-containing dressing materials in accordance with some example embodiments of the present invention. The graphs of FIGS. 6 and 7 show a summary of the results of how samples of the silver-containing dressing materials containing different amounts of silver performed at inhibiting bacterial growth, with a first set of samples that was exposed to gamma radiation (for sterilization and curing of the silicone composition) and a second set of samples that was sterilized using ethylene oxide. The results shown are for a qualitative in vitro susceptibility test used to demonstrate the release of an antimicrobial agent from a test article and the efficacy of the antimicrobial agent against a selected microorganism.

Testing was performed using the extended three-day zone of inhibition test where a 2.5 cm×2.5 cm dressing material sample was placed on a pre-inoculated agar plate with the microorganism under test. After an incubation period of 24 hours, zones of inhibition were determined, and the 2.5 cm×2.5 cm sample of the dressing material sample under test was transferred to a freshly prepared pre-inoculated agar plate of the microorganism under test. The two microorganisms selected were clinical isolates of pathogens associated with wound infection. The results show that the silver ions may be released from the dressing material sample, which may result in a zone of inhibition being formed around the dressing material sample. Greater antimicrobial activity may be shown with formulations of the dressing material having higher concentrations of the antimicrobial silver compound. Typically, with formulations of the dressing material having 2.5% or more silver material by weight of the silicone composition, the silver may be shown to be released over the three-day test period and to eradicate the microorganism. Additionally, samples that underwent gamma radiation having 2.5% or more silver material by weight of the silicone composition do not exhibit bacterial inhibition at levels significantly lower than samples that underwent ethylene oxide radiation. Additionally, samples having below 2.5% silver material also exhibit bacterial inhibition.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. A wound dressing product, comprising:
a substrate layer having an upper surface and a lower surface;
a tacky silicone coating composition present on said upper surface and on said lower surface;
a silver material disposed in the tacky silicone coating composition;
an upper release sheet adapted to cover the tacky silicone coating composition on the upper surface; and
a lower release sheet adapted to cover the tacky silicone coating composition on the lower surface.

2. The wound dressing product of claim 1, wherein the tacky silicone coating composition is hydrophobic, and does not include hydrophilic components.

3. The wound dressing product of claim 1, wherein the tacky silicone coating composition comprises a silicone prepolymer composition that has been cured thermally and by ionizing radiation.

4. The wound dressing product of claim 3, wherein the silver material is capable of inhibiting bacterial growth after undergoing exposure to ionizing radiation.

5. The wound dressing product of claim 4, wherein the ionizing radiation is gamma radiation.

6. The wound dressing product of claim 5, wherein the ability of the silver material to inhibit bacterial growth is not significantly reduced by gamma radiation compared to ethylene oxide sterilization.

7. The wound dressing product of claim 1, wherein the silver material is by weight 2.5% or more of the tacky silicone coating composition.

8. The wound dressing product of claim 1, wherein the upper surface is more tacky than the lower surface.

9. The wound dressing product of claim 1, wherein the lower release sheet is adapted to be removed from the lower surface more readily than the upper release sheet can be removed from the upper surface.

10. The wound dressing product of claim 1, wherein an array of apertures extends through the silicone coatings and the substrate layer.

11. The wound dressing product of claim 1, wherein the substrate layer comprises or consists essentially of a woven, nonwoven, or knitted mesh.

12. The wound dressing product of claim 1, wherein the tackiness of the upper surface is from 20% to 100% greater than the tackiness of the lower surface.

13. The wound dressing product of claim 1, wherein the silicone on the upper and lower surfaces is substantially chemically homogenous.

14. The wound dressing product of claim 1, wherein the silver material comprises silver acetate.

15. The wound dressing product of claim 1 which is sterile and packaged in a microorganism-impermeable container.

16. A method of making a wound dressing material, comprising:
providing a substrate layer having an upper surface and a lower surface;
coating the upper surface and the lower surface of the substrate layer with a fluid silicone prepolymer composition comprising a silver material;
thermally partially curing the silicone prepolymer composition to produce an intermediate material having a partially-cured silicone composition on the upper surface and the lower surface; and
further curing the partially-cured silicone composition by exposing the intermediate material to ionizing radiation, to produce a final material having tacky silicone coatings on the upper surface and the lower surface;
wherein the step of coating applies unequal weights of the silicone prepolymer composition to the upper surface and the lower surface.

17. The method of claim 16, further comprising storing the intermediate material at a temperature below 50° C. for at least two days to allow equilibration of the silicone coatings on the upper surface and the lower surface.

18. The method of claim 17, wherein the step of storing is carried out for a time of from two weeks to ten weeks.

19. The method of claim 16, wherein said fluid silicone prepolymer composition comprises:
a vinyl functional polydimethylsiloxane;
a hydrogen functional siloxane;
a hydrosilylation catalyst; and
a polymerization inhibitor adapted to be evaporated from the composition during the step of thermally partially curing.

20. The method of claim 16, further comprising applying an upper release sheet and a lower release sheet over the silicone composition on the upper surface and the lower surface.

21. The method of claim 20, wherein the upper release sheet and the lower release sheet are applied intermediate the steps of thermally partially curing and storing.

22. The method of claim 16, wherein the silver material comprises silver acetate.

23. The method of claim 16, further comprising the step of packaging the intermediate material in a microorganism-impermeable container prior to the step of further curing, and wherein the step of further curing also sterilizes the material.

24. The method of claim 16, wherein the tacky silicone coatings are hydrophobic, and do not include hydrophilic components.

25. The method of claim 16, wherein the silver material is capable of inhibiting bacterial growth after exposure to the ionizing radiation.

26. The method of claim 16, wherein the ionizing radiation is gamma radiation.

27. The method of claim 16, wherein the silver material is by weight 2.5% or more of the tacky silicone coatings.

28. A method of making a wound dressing material, comprising:
providing a substrate layer having an upper surface and a lower surface;
coating the upper surface and the lower surface of the substrate layer with a fluid silicone prepolymer composition comprising a silver material;
thermally partially curing the silicone prepolymer composition to produce an intermediate material having a partially-cured silicone composition on the upper surface and the lower surface; and
further curing the partially-cured silicone composition by exposing the intermediate material to ionizing radiation, to produce a final material having tacky silicone coatings on the upper surface and the lower surface;
wherein the step of thermally partially curing applies different amounts of heat to the upper surface and the lower surface, whereby the silicone coatings on the upper surface and the lower surface have different tackiness following the further curing step.

29. The method of claim 28, wherein the silver material comprises silver acetate.

30. The method of claim 28, wherein the tacky silicone coatings are hydrophobic, and do not include hydrophilic components.

31. The method of claim 28, wherein the silver material is capable of inhibiting bacterial growth after exposure to the ionizing radiation.

32. The method of claim 28, wherein the ionizing radiation is gamma radiation.

33. The method of claim 28, wherein the silver material is by weight 2.5% or more of the tacky silicone coatings.

34. The systems, apparatuses, and methods substantially as described herein.
